(19) United States
(12) Patent Application Publication
(10) Pub. No.: US 2008/0161927 A1
(43) Pub. Date: Jul. 3, 2008

(54) Intervertebral Implant with Porous Portions

(75) Inventors: Heather Savage, Memphis, TN (US); Anthony J. Melkent, Memphis, TN (US); Jason Allan Edie, Memphis, TN (US); Jeffrey Rouleau, Maple Grove, MN (US); Frank Bono, Collierville, TN (US); John L. White, Bartlett, TN (US)

Correspondence Address: HAYNES AND BOONE, LLP, 901 Main Street, Suite 3100, Dallas, TX, 75202, US

(73) Assignee: WARSAW ORTHOPEDIC, INC., Warsaw, IN (US)

(21) Appl. No.: 11/550,479

(22) Filed: Oct. 18, 2006

(51) Int. Cl. A61F 2/44

(52) U.S. Cl. 623/17.16

(57) ABSTRACT

A device for promoting fusion of first and second vertebrae, comprises a first solid region formed of non-porous polyetheretherketone (PEEK) and a first porous region including a porous PEEK architecture. The first porous region is bonded to the first solid region.

### BACKGROUND

Spinal discs that extend between the endplates of adjacent vertebrae in a spinal column of the human body provide support to the adjacent vertebrae and enable motion in the vertebral joint. These discs can rupture, degenerate and/or protrude by injury, degradation, disease to such a degree that the intervertebral space between adjacent vertebrae collapses as the disc loses at least a part of its support function. This can cause impingement of the nerve roots and severe pain. In some cases, surgical correction may be required.

The surgical correction may include an interbody fusion procedure wherein the disc is at least partially excised and an implant placed within the space previously occupied by the disc material to restore a more normal spatial relationship. The fusion procedure may provide stability in the short term through mechanical support and in the long term by the permanent cross bonding of bone from vertebra to vertebra. There is a need for improved implant designs which will further enhance both the short and long term stability of the implant and promote fusion of the joint.

### SUMMARY

In one embodiment, a device for promoting fusion of first and second vertebrae, comprises a first solid region formed of non-porous polyetheretherketone (PEEK) and a first porous region including a porous PEEK architecture. The first porous region is bonded to the first solid region.

In another embodiment, a surgical method comprises removing at least a portion of an intervertebral disc from between a pair of vertebral bodies to create an intervertebral space. The method further includes selecting an a device comprising a solid region including a non-porous polymer and a porous region including a porous polymer architecture. The porous region is bonded to the solid region. The method further includes inserting the device into the intervertebral space to promote bone growth between the pair of vertebral bodies.

In another embodiment, an apparatus comprises a first wall adapted to engage a first vertebral endplate, a second wall adapted to engage a second vertebral endplate, a third wall extending generally orthogonally between the first and second walls, and a fourth wall extending generally orthogonally between the first and second walls and spaced apart from the third wall. The apparatus further includes a cavity defined at least in part by the first, second, third, and fourth walls and adapted to receive a bone growth promoting material. At least one of the walls includes a solid region formed of non-porous first material and a scaffold region formed of a porous second material.

In another embodiment, an apparatus for promoting bone growth between first and second bone portions comprises a first solid layer formed of non-porous PEEK and a first porous layer formed of PEEK particles. The PEEK particles are sintered together to form the first porous layer, and the PEEK particles are fused to the non-porous PEEK of the first solid layer such that the first porous layer covers at least a portion of the first solid layer.

Additional embodiments are included in the attached drawings and the description provided below.

### BRIEF DESCRIPTION OF THE DRAWINGS

FIG. 1 is a sagittal view of a section of a vertebral column.

FIG. 2 is a perspective view of an intervertebral implant with porous segments according to one embodiment of the present disclosure.

FIG. 3 is a side view of the intervertebral implant according to the embodiment of FIG. 2.

FIG. 4 is a top view of the intervertebral implant according to the embodiment of FIG. 2.

FIGS. 5 and 6 are top views of an intervertebral implants according to alternative embodiments of the present disclosure.

FIG. 7 is a side view of an intervertebral implant according to an alternative embodiment of the present disclosure.

FIG. 8 is a top view of the intervertebral implant according to the embodiment of FIG. 7.

FIG. 9 is a side view of an intervertebral implant according to an alternative embodiment of the present disclosure.

FIG. 10 is a top view of the intervertebral implant according to the embodiment of FIG. 9.

FIG. 11 is a side view of an intervertebral implant according to an alternative embodiment of the present disclosure.

FIG. 12 is a top view of the intervertebral implant according to the embodiment of FIG. 11.

FIG. 13 is a side view of an intervertebral implant according to an alternative embodiment of the present disclosure.

FIG. 14 is a top view of the intervertebral implant according to the embodiment of FIG. 13.

FIG. 15 is a side view of an intervertebral implant according to an alternative embodiment of the present disclosure.

FIG. 16 is a top view of the intervertebral implant according to the embodiment of FIG. 15.

FIG. 17 is a perspective view of a crescent-shaped intervertebral implant according to an alternative embodiment of the present disclosure.

FIG. 18 is a perspective view of a crescent-shaped intervertebral implant according to an alternative embodiment of the present disclosure.

FIG. 19 is a side view of a boomerang-shaped layered intervertebral implant according to an alternative embodiment of the present disclosure.

FIG. 20 is a top view of a boomerang-shaped intervertebral implant according to the embodiment of FIG. 90.

FIG. 21 is a side view of the intervertebral implant according to the another embodiment of the present disclosure.

FIG. 22 is a cross-sectional side view of an intervertebral implant according to another embodiment of the present disclosure.

FIG. 23 is a top view of a ring shaped intervertebral implant according to another embodiment of the present disclosure.

FIG. 24 is a cross-sectional view of the ring shaped intervertebral implant according to the embodiment of FIG. 23.

FIG. 25 is a top view of a ring shaped intervertebral implant according to another embodiment of the present disclosure.

FIG. 26 is a cross-sectional view of the ring shaped intervertebral implant according to the embodiment of FIG. 25.

FIG. 27 is a side view of a circular shaped intervertebral implant according to another embodiment of the present disclosure.

FIG. 28 is a top view of the circular shaped intervertebral implant according to the embodiment of FIG. 27.

FIG. 29 is a perspective view of a boomerang shaped intervetebral implant according to another embodiment of the present disclosure.

FIG. 30 is a top view of a boomerang shaped intervertebral implant according to another embodiment of the present disclosure.

FIG. 31 is a cross-sectional view of the intervertebral implant according to the embodiment of FIG. 30.

FIG. 32 is a top view of a boomerang shaped intervertebral implant according to another embodiment of the present disclosure.

FIG. 33 is a cross-sectional view of the intervertebral implant according to the embodiment of FIG. 32.

FIG. 34 is a top view of a boomerang shaped intervertebral implant according to another embodiment of the present disclosure.

FIG. 35 is a cross-sectional view of the intervertebral implant according to the embodiment of FIG. 34.

FIG. 36 is a top view of a boomerang shaped intervertebral implant according to another embodiment of the present disclosure.

FIG. 37 is a cross-sectional view of the intervertebral implant according to the embodiment of FIG. 36.

FIG. 38 is a perspective view of an intervertebral implant according to another embodiment of the present disclosure.

FIG. 39 is a side view of an intervertebral implant according to another embodiment of the present disclosure.

FIG. 40 is a cross-sectional view of an intervertebral implant according to the embodiment of FIG. 39.

FIG. 41 is a side view of an intervertebral implant according to another embodiment of the present disclosure.

FIG. 42 is a cross-sectional view of an intervertebral implant according to the embodiment of FIG. 41.

FIG. 43 is a side view of an intervertebral implant according to another embodiment of the present disclosure.

FIG. 44 is a cross-sectional view of an intervertebral implant according to the embodiment of FIG. 43.

FIG. 45 is a side view of an intervertebral implant according to another embodiment of the present disclosure.

FIG. 46 is a cross-sectional view of an intervertebral implant according to the embodiment of FIG. 45.

FIG. 47 is a perspective view of an intervertebral implant according to another embodiment of the present disclosure.

FIG. 48 is a top view of an intervertebral implant according to the embodiment of FIG. 47.

FIG. 49 is a cross-sectional view of an intervertebral implant according to the embodiment of FIG. 47.

FIG. 50 is a top view of an intervertebral implant according to another embodiment of the present disclosure.

FIG. 51 is a cross-sectional view of an intervertebral implant according to the embodiment of FIG. 50.

FIG. 52 is a top view of an intervertebral implant according to another embodiment of the present disclosure.

FIG. 53 is a cross-sectional view of an intervertebral implant according to the embodiment of FIG. 52.

FIG. 54 is a top view of an intervertebral implant according to another embodiment of the present disclosure.

FIG. 55 is a cross-sectional view of an intervertebral implant according to the embodiment of FIG. 54.

FIG. 56 is a top view of an intervertebral implant according to another embodiment of the present disclosure.

FIG. 57 is a cross-sectional view of an intervertebral implant according to the embodiment of FIG. 56.

FIG. 58 is a top view of an intervertebral implant according to another embodiment of the present disclosure.

FIG. 59 is a cross-sectional view of an intervertebral implant according to the embodiment of FIG. 58.

FIG. 60 is a perspective view of an intervertebral implant according to another embodiment of the present disclosure.

FIG. 61 is a side view of an intervertebral implant according to the embodiment of FIG. 60.

FIG. 62 is a perspective view of an intervertebral implant according to another embodiment of the present disclosure.

FIG. 63 is a side view of an intervertebral implant according to the embodiment of FIG. 62.

FIG. 64 is an exploded perspective view of an intervertebral implant according to another embodiment of the present disclosure.

FIG. 65 is a perspective view of an intervertebral implant according to another embodiment of the present disclosure.

FIG. 66 is a perspective view of an intervertebral implant according to another embodiment of the present disclosure.

FIG. 67 is a perspective view of an intervertebral implant according to another embodiment of the present disclosure.

### DETAILED DESCRIPTION

The present disclosure relates generally to devices and methods for promoting bone growth between portions of bone and, more particularly, to devices and methods for fusing adjacent vertebral bodies. For the purposes of promoting an understanding of the principles of the invention, reference will now be made to the embodiments, or examples, illustrated in the drawings and specific language will be used to describe the same. It will nevertheless be understood that no limitation of the scope of the invention is thereby intended. Any alterations and further modifications in the described embodiments, and any further applications of the principles of the invention as described herein are contemplated as would normally occur to one skilled in the art to which the invention relates.

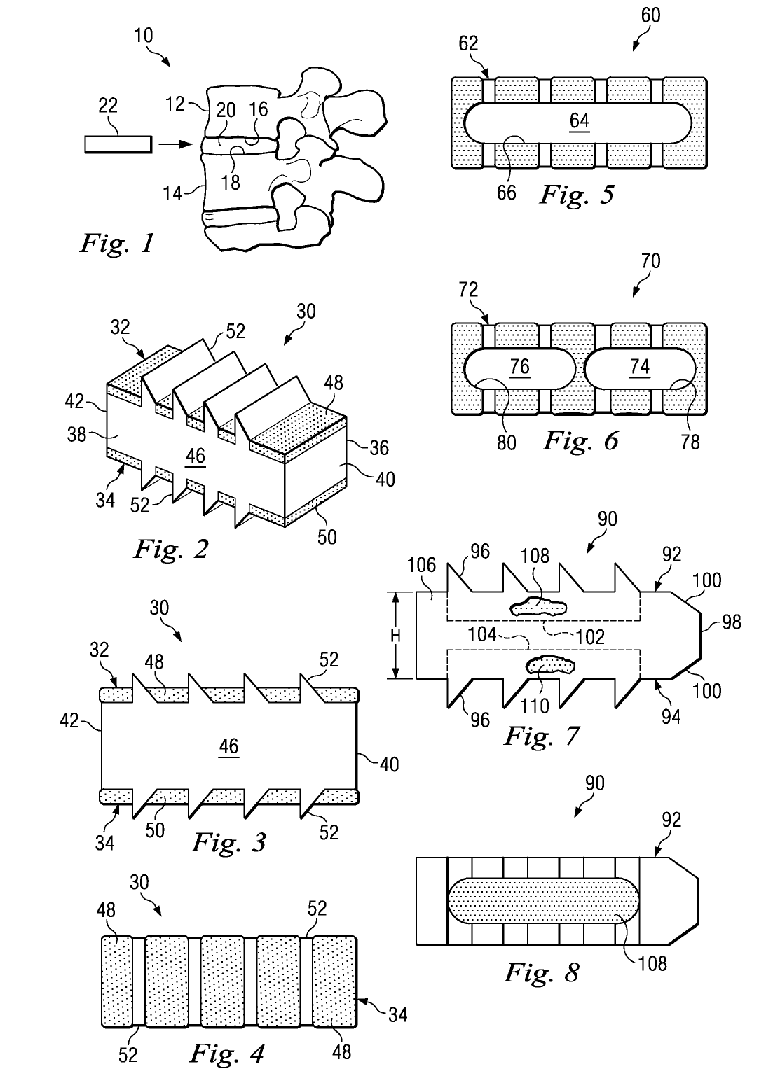

Referring first to FIG. 1, the reference numeral 10 refers to a vertebral joint section or a motion segment of a vertebral column. The joint section 10 includes adjacent vertebral bodies 12, 14. The vertebral bodies 12, 14 include end plates 16, 18, respectively. An intervertebral disc space 20 is located between the endplates 16, 18. A device or apparatus 22 may be inserted into the intervertebral disc space 20 to promote fusion or preserve motion between the vertebral bodies 12, 14. Although the device 22 is shown to be entering the intervertebral disc space through an anterior approach, it is understood that posterior, posterolateral, or anterolateral approaches may also be suitable.

Referring now to FIGS. 2-4, in one embodiment the device 22 may be an intervertebral fusion implant device referred to by the reference numeral 30. The device 30 includes an upper wall 32 and a lower wall 34. Side walls 36, 38 extend in a generally orthogonal direction from the upper and lower walls 32, 34. Side walls 40, 42 extend generally orthogonally from the side walls 36, 38. In this embodiment, the upper and lower walls, 32, 34 are generally parallel to one another; the side walls 36, 38 are generally parallel to one another; and the side walls 40, 42 are generally parallel to one another.

Referring still to FIGS. 2-4, the device 30 includes a solid region or layer 46 bonded to porous regions or layers 48, 50. The solid layer 46 may be formed of biocompatible materials including metals, polymers, ceramics, or composite materials. Specifically, suitable materials may include metals such as cobalt-chromium alloys, titanium alloys, nickel titanium alloys, magnesium alloys, and/or stainless steel alloys. Suitable polymer materials may inlcude any member of the polyaryletherketone (PAEK) family such as polyetheretherketone (PEEK), carbon-reinforced PEEK, or polyetherketoneketone (PEKK); polysulfone; polyetherimide; polyimide; ultra-high molecular weight polyethylene (UHMWPE); and/or cross-linked UHMWPE. Relatively solid ceramic materials such as aluminum oxide or alumnia, zirconium oxide or zirconia, compact of particulate diamond, and/or pyrolytic carbon may be suitable. The solid layer 46 may be a uniform mass of material or may be a composite material having low or no porosity. A relative absence of voids throughout the solid layer 46 may serve to limit or block bone in-growth. The solid layer 46 may provide sufficient rigidity and structural integrity to substantially maintain the height of the intervertebral space 20 between the vertebral bodies 12, 14, and to withstand any internal or external forces applied to the spinal joint 10 of which the vertebral bodies 12, 14 are a part.

The porous layers 48, 50 may also be formed of biocompatible materials including metals, polymers, ceramics, or composite materials, including the specific materials listed above. The biocompatible materials of the porous layers may, however, be suspended in a porous architecture such that interconnected voids are interspersed between particles of the material. For example, in one embodiment, the porous layer may be formed of PEEK particles including spherical and/or non-spherical beads. The beads may be uniformly or randomly sized. The PEEK particles may be sintered, using laser, heat or ultrasonic processes, to form a sintered mass such that the PEEK particles occupy approximately 50-75% of the volume of the sintered mass, although higher or lower porosities may also be suitable. In alternative embodiments, a porous layer may be created through the use of foaming agents, chemical etching, plasma spray, electrical discharge machining, drilling, or other types of void creation techniques. Generally, the porous layer may serve as a scaffold, permitting bone in-growth and fusion.

The porous layers 48, 50 may be molded, machined, or otherwise formed into a desired shape and size. Once formed, the porous layer 48, 50 may then be bonded or fused to a surface of the solid layer 46 using heat treatment, a chemical adhesive, or any other bonding technique known in the art. The porous layers 48, 50 may, alternatively, be formed in situ with sprays, coatings, or other material deposition techniques applied directly to a surface of the solid region. Although the porous layers may formed into any thickness desired to achieve the objective of the device 30, in one embodiment, the porous layers may have a thickness between 0.5 and 3.0 millimeters.

Referring again to FIGS. 2-4, a series of projections 52 extend from the upper and lower walls 32, 34, respectively. The projections 52 may be integrally formed with and project from the solid layer 46 to provide both initial and long term stability to the implanted device 30. In this embodiment, the projections 52 are generally triangular as viewed from a side profile and extend across the upper and lower walls 32, 34. In alternative embodiments, the projections may take other suitable shapes including keels, pyramids, cones, and spikes.

Although the embodiment of FIGS. 2-4 has generally parallel opposite walls, in alternative embodiments, the upper and lower walls 32, 34 may be angled relative to one another to achieve a desired kyphosis or lordosis. In still other embodiments, any of the walls may be curved. In still other embodiments, a wall may extend obliquely from an adjacent wall rather than orthogonally. In still other alternative embodiments, the device may include geometry for mating with insertion or revision instruments. Furthermore, at least one of the walls may be tapered or bullet-nosed to provide a self-distraction useful during insertion of the device.

Prior to implantation of the intervertebral fusion device 30, the disc space 20 may be prepared with a partial or complete discectomy. Portions of the vertebral bodies 12, 14, and/or vertebral endplates 16, 18 may be resected, machined, or otherwise prepared to fit the shape of the device 30 and to promote subsequent tissue growth. With the disc space 20 prepared, the physician may select and insert the device 30 into the disc space 20 such that the upper wall 32 is positioned adjacent to the vertebral endplate 16 and the lower wall 34 is positioned adjacent to the vertebral endplate 18. The projections 52 may engage the vertebral endplates 16, 18 to provide stability to the implanted device 30. As implanted, the porous layer 48 may contact the vertebral endplate 16, and the porous layer 50 may contact the vertebral endplate 18. While the solid layer 46 may serve a primarily load-bearing function, the porous layers 48, 50 may permit bone in-growth, eventually resulting in a fusion of the joint 10. The implantation procedure may be approached from an anterior, posterior, or any suitable oblique direction.

Referring now to FIG. 5, according to another embodiment, an intervertebral fusion device 60 may be substantially similar to the device 30 described above with the differences to be noted. The device 60 includes an upper wall 62 through which an elongated opening 64 extends. The opening 64 may further extend through a lower wall in the device 60. The opening 64 may form a through passage 66 through the solid layer such that non-porous material defines the passage. Alternatively, the passage may be lined with a porous layer similar to layers 48, 50. The porous layers of the upper wall 62 may wrap into the opening 64, if desired.

Prior to or during implantation of the device 60 into the intervertebral space 20, the passage 66 may be filled with a filler material (not shown). The filler material may be composed of any type of material that has the ability to promote, enhance and/or accelerate the growth of new bone tissue by one or more mechanisms such as, for example, osteogenesis, osteoconduction and/or osteoinduction, including, for example, allograft chips, bone marrow, a demineralized bone matrix putty or gel and/or any combination thereof. Together with the porous layers of the device 60, the filler material may promote bone growth through and around the device to promote fusion of the joint 10. It is understood that in this embodiment, and in those below that describe the use of filler material, filler material is optional and may be omitted.

Referring now to FIG. 6, according to another embodiment, an intervertebral fusion device 70 may be substantially similar to the device 60 described above with the differences to be noted. The device 70 includes an upper wall 72 through which two elongated openings 74, 76 extend. The openings 74, 76 may further extend through a lower wall in the device 70. The openings 74, 76 may form through passages 78, 80, respectively, through the solid layer such that non-porous material defines the passage. It is understood that more than two openings may be similarly formed. Alternatively, the passage may be lined with a porous layer similar to layers 48, 50. As described above, prior to or during implantation of the device 70 into the intervertebral space 20, the passages 78, 80 may be filled with a filler material (not shown) to promote bone ingrowth and fusion.

Referring now to FIGS. 7 and 8, according to another embodiment, an intervertebral fusion device 90 may be substantially similar to the device 60 described above with the differences to be noted. The device 90 includes an upper wall 92 and a lower wall 94. Projections 96 extend from the upper and lower walls 92, 94. A side wall 98, which may be a leading wall as installed, includes tapered portions 100 to provide distraction useful for implantation. The device 90 includes recessed walls 102, 104 which in this embodiment may have an oblong shape as shown in the top view of FIG. 8. The recessed walls 102, 104 are recessed less than half of the height H of the device 90 and into the upper and lower walls, 92, 94, respectively. As shown in FIG. 7, a solid region 106 extends through the device 90 and between the recessed walls 102, 104. In this embodiment, a porous layer may be omitted from the upper and lower walls, 92, 94. However, porous layers 108, 110 may extend across the recessed walls 102, 104, respectively and become bonded to the solid region 106.

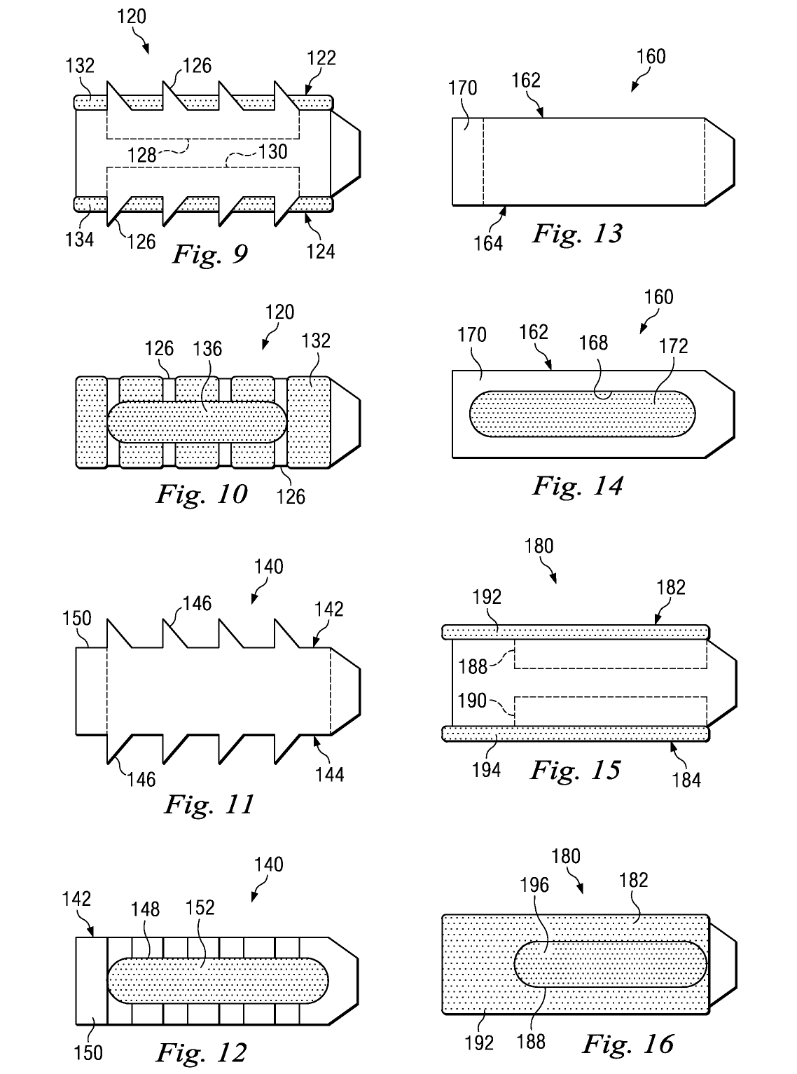

Referring now to FIGS. 9 and 10, according to another embodiment, an intervertebral fusion device 120 may be substantially similar to the device 90 described above with the differences to be described below. The device 120 includes an upper wall 122 and a lower wall 124. Projections 126 extend from the upper and lower walls 122, 124. The device 120 includes recessed walls 128, 130 which in this embodiment may have an oblong shape as shown in the top view of FIG. 10. In this embodiment, the upper wall 122 includes a porous portion 132, and the lower wall 124 includes a porous portion 134. The porous portions 132, 134 may be substantially similar to the porous portions 48, 50 described above. The recessed wall 128 may include a porous layer 136 which may extend continuously into the porous portion 132. In alternative embodiments, the porous layer 136 may be discontinuous from the porous portion 132. The projections 126 may extend through the porous portions 132, 134.

Referring now to FIGS. 11 and 12, according to another embodiment, an intervertebral fusion device 140 may be substantially similar to the device 90 described above with the differences to be noted. The device 140 includes an upper wall 142 and a lower wall 144. Projections 146 extend from the upper and lower walls 142, 144. The device 140 includes an aperture 148, which in this embodiment may be oblong as shown in the top view of FIG. 12. The aperture 148 extends from the upper wall 142, through the lower wall 144, and through a solid region 150 which may be substantially similar to the solid region 46 described above. The aperture 148 may be filled with a porous layer 152 which may be substantially similar to the porous layer 48 described above. In this embodiment, the porous layer 152 may promote bone growth through the aperture 148 and thus through the implant 140 to fuse the joint 10.

Referring now to FIGS. 13 and 14, according to another embodiment, an intervertebral fusion device 160 may be substantially similar to the device 140 described above with the differences to be noted. The device 160 includes an upper wall 162 and a lower wall 164. In this embodiment, however, projections are omitted. The device 160 includes an aperture 168, which in this embodiment may be oblong as shown in the top view of FIG. 14. The aperture 168 extends from the upper wall 162, through the lower wall 164, and through a solid region 170 which may be substantially similar to the solid region 46 described above. The aperture 168 may be filled with a porous layer 172 which may be substantially similar to the porous layer 48 described above. In this embodiment, the porous layer 172 may promote bone growth through the aperture 168 and thus through the implant 160 to fuse the joint 10.

Referring now to FIGS. 15 and 16, according to another embodiment, an intervertebral fusion device 180 may be substantially similar to the device 120 described above with the differences to be noted. The device 180 includes an upper wall 182 and a lower wall 184. In this embodiment, projections are omitted. The device 180 includes recessed walls 188, 190 which in this embodiment may be oblong as shown in the top view of FIG. 16. In this embodiment, the upper wall 182 includes a porous portion 192, and the lower wall 184 includes a porous portion 194. The porous portions 192, 194 may be substantially similar to the porous portions 48, 50 described above. The recessed wall 188 may include a porous layer 196 which may extend continuously into the porous portion 192. In alternative embodiments, the porous layer 196 may be discontinuous from the porous portion 192.

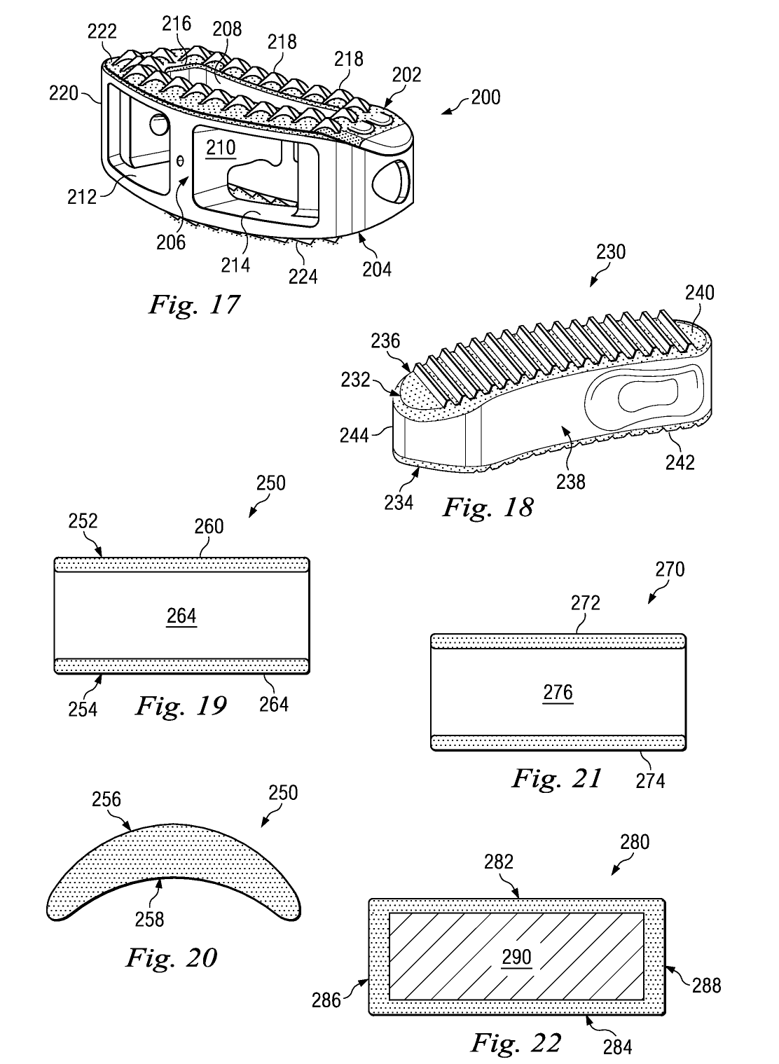

Referring now to FIG. 17, in another embodiment the intervertebral device 22 may be an intervertebral fusion device referred to by the reference numeral 200. The device 200 may have a similar geometry to the BOOMERANG® or CRESCENT® vertebral spacers (distributed by or in development with Medtronic, Inc. of Minneapolis, Minn.) as described, at least in part, in U.S. Pat. Nos. 6,764,491; 6,830,570; and 6,447,547, which are incorporated by reference herein.

The device 200 includes an upper wall 202 and a lower wall 204, both of which have a crescent or boomerang shape. A convexly curved side wall 206 extends generally orthogonally between the upper wall 202 and the lower wall 204. A concavely curved side wall 208 extends generally orthogonally between the upper wall 202 and the lower wall 204. Although the upper and lower walls are shown to be generally parallel, it is understood that lordotic or kyphotic configurations may also be suitable. Furthermore, the upper and lower walls may be convex, bi-convex, or concave.

A bracing wall 210 extends between the side walls 206, 208. Apertures 212, 214 extend through the side walls 206, 208. An aperture 216 extends through the upper wall 202 and the lower wall 204. Pyramidal projections 218 may extend from the upper and lower walls 202, 204. The upper, lower and side walls 202, 204, 206, 208 may be tapered or bullet-nosed to provide a self-distraction useful during insertion of the device.

The device 200 includes a solid region or layer 220 bonded to porous regions or layers 222, 224. The solid layer 220 may be formed of any of the biocompatible materials listed above for solid layer 46, including metals, polymers, ceramics, or composite materials. The solid layer 220 may be a uniform mass of material or may be a composite material having low or no porosity. Generally, the solid layer 220 may provide durability and/or strength to the device 200.

The porous layers 222, 224 may also be formed of biocompatible materials including metals, polymers, ceramics, or composite materials, including the specific materials listed above for porous layers 48, 50. The biocompatible materials of the porous layers are suspended in a porous architecture such that interconnected voids are interspersed between particles of the material. Generally, the porous layer may serve as a scaffold, permitting bone in-growth and fusion. The porous layers 222, 224 may be formed and bonded to the solid layer 220 as described above for layers 48, 50. In this embodiment, the solid layer 220 forms the side walls 206, 208 and the bracing wall 210. In alternative embodiments, however, all or portions of the side and bracing walls may be formed of porous regions. The device 200 may be implanted using the methods described above for device 30. After the insertion of the device 200 between the vertebral bodies 12, 14 has been completed, the device 200 may promote the fusion or joining together of the vertebral bodies 12, 14.

In alternative embodiments, the upper and lower walls may be angled relative to one another to achieve a desired kyphosis, lordosis, or lateral wedge effect. In still other embodiments, a wall may extend obliquely from an adjacent wall rather than orthogonally. In still other alternative embodiments, the device may include geometry for mating with insertion or revision instruments.

Referring now to FIG. 18, in another embodiment the intervertebral device 22 may be a device 230 substantially similar to the device 200 with the differences noted below. The device 230 may have a similar geometry to the BOOMERANG® or CRESCENT® vertebral spacers (distributed by or in development with Medtronic, Inc. of Minneapolis, Minn.) as described, at least in part, in detail in U.S. Pat. Nos. 6,764,491; 6,830,570; and 6,447,547, which are incorporated by reference herein.

The device 230 includes an upper wall 232 and a lower wall 234, both of which have a crescent or boomerang shape. A convexly curved side wall 236 and a concavely curved side wall 238 may extend between the upper and lower walls 232, 234. The upper and lower walls 232, 234 may include porous layers 240, 242, respectively. A solid region 244 extends between the porous layers 240, 242.

Referring now to FIGS. 19 and 20, in another embodiment, a device 250 is substantially similar to the device 230. The device 250 includes an upper wall 252 and a lower wall 254, both of which have a crescent or boomerang shape. A convexly curved side wall 256 and a concavely curved side wall 258 may extend between the upper and lower walls 252, 254. The upper and lower walls 232, 234 may include porous layers 260, 262, respectively. A solid region 264 extends between the porous layers 260, 262.

Referring now to FIG. 21, in another embodiment the intervertebral device 22 may be a device 270 substantially similar to the device 250. The device 270 may include upper and lower porous layers 272, 274, respectively. A solid region 276 extends between the porous layers 272, 274. This basic layered configuration may be used with devices having a variety of top view configurations including circular, oval, oblong, bean shaped, D-shaped, rectangular, or any other shape to promote fusion between the vertebral bodies 12, 14 and provide surgical ease of use. A variety of side view configurations as viewed from perpendicular to the view in FIG. 21 may include circular, rectangular, or wedge shaped.

Referring now to FIG. 22, in another embodiment the intervertebral device 22 may be a device 280 substantially similar to the device 270, with the exceptions noted below. The device 280 may include upper and lower porous layers 282, 284, respectively. The device 280 may also include side porous layers 286, 288. A solid region 290 extends between the porous layers 282, 284, 286, 288 such that the solid region 290 is encapsulated by porous layers. This basic encapsulated configuration may be used with devices having a variety of top view configurations including circular, oval, oblong, bean shaped, D-shaped, rectangular, or any other shape to promote fusion between the vertebral bodies 12, 14 and provide surgical ease of use. A variety of side view configurations as viewed from perpendicular to the view in FIG. 22 may include circular, rectangular, or wedge shaped.

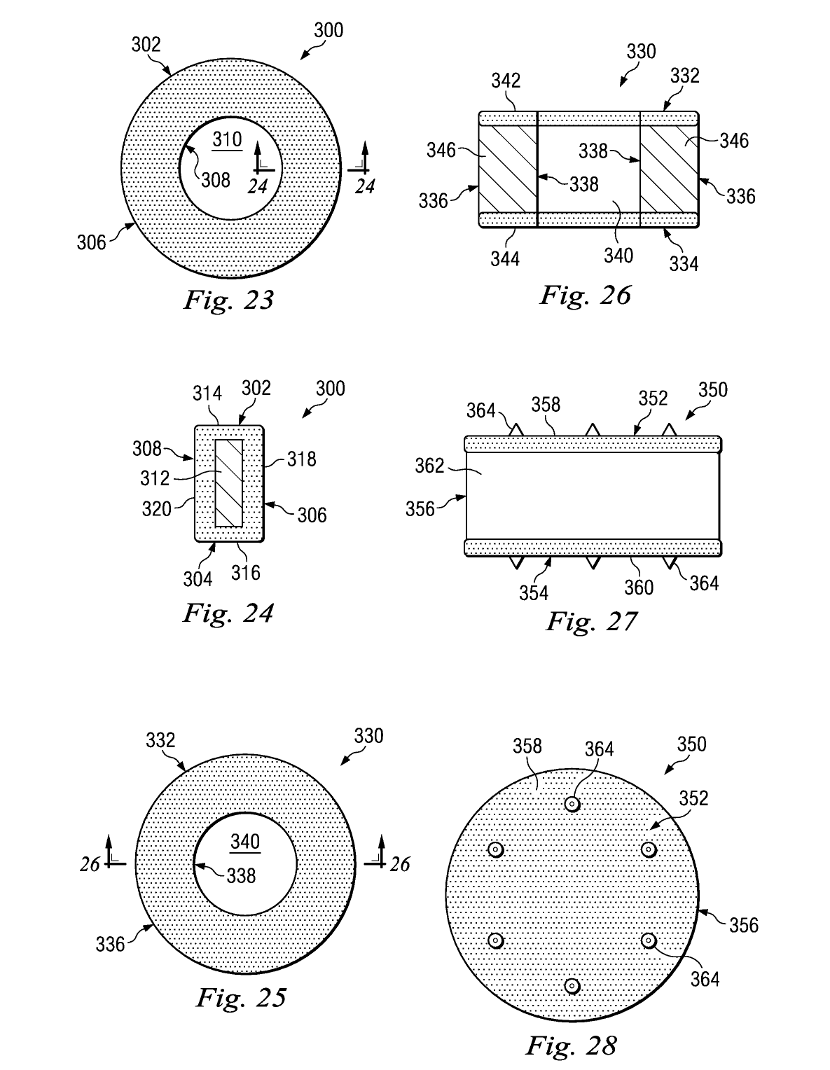

Referring now to FIGS. 23 and 24, in another embodiment the intervertebral device 22 may be an intervertebral fusion device referred to by the reference numeral 300. The device 300 may be cylindrical and include an upper wall 302 and a lower wall 304. An outer side wall 306 forms the outer perimeter of the cylindrical device 300 and extends generally orthogonally between the upper wall 302 and the lower wall 304. An inner side wall 308 forms the inner perimeter of the cylindrical device 300 and extends generally orthogonally between the upper wall 302 and the lower wall 304. The side wall 308 defines a through passage 310.

The device 300 includes a solid region or layer 312 bonded to upper and lower porous regions 314, 316 and to side porous regions 318, 320. The solid layer 312 may be formed of any of the biocompatible materials listed above for solid layer 46, including metals, polymers, ceramics, or composite materials. The solid layer 312 may be a uniform mass of material or may be a composite material having low or no porosity. Generally, the solid layer 312 may provide durability and/or strength to the device 300.

The porous layers 314, 316, 318, 320 may also be formed of biocompatible materials including metals, polymers, ceramics, or composite materials, including the specific materials listed above for porous layers 48, 50. The biocompatible materials of the porous layers are suspended in a porous architecture such that interconnected voids are interspersed between particles of the material. Generally, the porous layer may serve as a scaffold, permitting bone in-growth and fusion. The porous layers 314, 316, 318, 320 may be formed and bonded to the surfaces of the solid layer 312 as described above for layers 48, 50. In this embodiment, the solid layer 312 is encapsulated by the porous layers 314, 316, 318, 320. The device 300 may be implanted using the methods described above for device 30. The passage 310 may be filled with bone graft or other bone growth material as described above to promote tissue growth through the passage 310. After the insertion of the device 300 between the vertebral bodies 12, 14 has been completed, the device 300 may promote the fusion or joining together of the vertebral bodies 12, 14.

Referring now to FIGS. 25 and 26, in another embodiment the intervertebral device 22 may be a device 330 substantially similar to the device 300, with the differences noted below. The device 330 may also be cylindrical and include an upper wall 332 and a lower wall 334. An outer side wall 336 forms the outer perimeter of the cylindrical device 330 and extends generally orthogonally between the upper wall 332 and the lower wall 334. As inner side wall 338 forms the inner perimeter of the cylindrical device 330 and extends generally orthogonally between the upper wall 332 and the lower wall 334. The side wall 338 defines a through passage 340. In this embodiment, upper and lower walls 332, 334 include porous regions 342, 344. The porous regions 332, 334 are bonded to upper and lower surfaces of a solid region 346. In this embodiment, the solid region 346 extends from the inner side wall 338 to the outer side wall 336, forming a layered configuration with no porous regions along the side walls. Like the device 300, the device 330 is adapted to promote fusion of the joint 10 by promoting bone growth through the passage 340 and into the porous regions 342, 344.

Referring now to FIGS. 27 and 28, in another embodiment the intervertebral device 22 may be a device 350 substantially similar to the device 330, with the differences noted below. The device 350 may also be cylindrical and include an upper wall 352 and a lower wall 354. An outer side wall 356 forms the outer perimeter of the cylindrical device 350 and extends generally orthogonally between the upper wall 352 and the lower wall 354. In this embodiment, a through passage is omitted and walls 352, 354 extend continuously across the diameter of the circle defined by the outer side wall 356. In this embodiment, upper and lower walls 352, 354 include porous regions 358, 360. The porous regions 358, 360 are bonded to upper and lower surfaces of a solid region 362. In this embodiment, the solid region 362 extends continuously across the diameter of the circle defined by the outer side wall 356. The resulting structure is a layered configuration with no porous regions along the side wall. Projections 364 extend from the upper and lower walls 352, 354 and through the porous regions 358, 360. The projections may be integrally formed with the solid region 362 or may be assembled to the solid region 362. In an alternative embodiment, porous regions may be bonded to the side wall. In still another alternative embodiment, a through hole may be formed in the device 350.

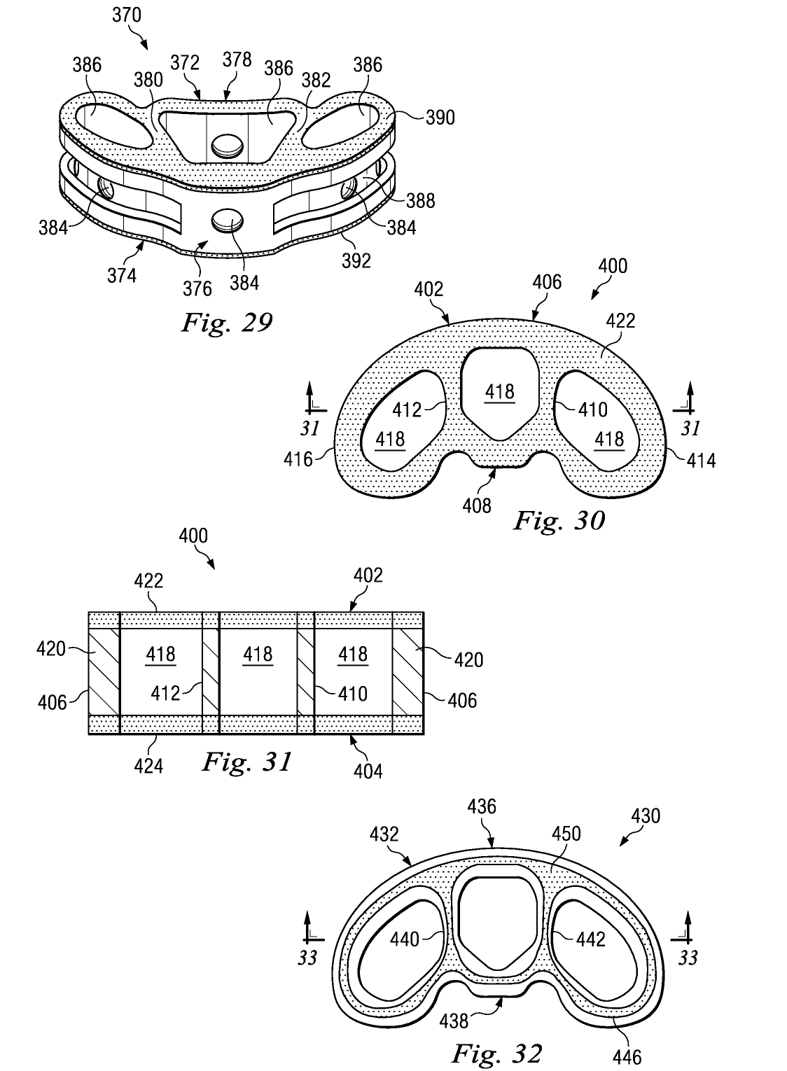

Referring now to FIG. 29, in another embodiment the intervertebral device 22 may be an intervertebral fusion device referred to by the reference numeral 370. The device 370 may have a similar geometry to the BOOMERANG® or CRESCENT® vertebral spacers (distributed by or in development with Medtronic, Inc. of Minneapolis, Minn.) as described, at least in part, in U.S. Pat. Nos. 6,764,491; 6,830,570; and 6,447,547, which are incorporated by reference herein.

The device 370 includes an upper wall 372 and a lower wall 374, both of which have a generally crescent or boomerang shape. A convexly curved side wall 376 extends generally orthogonally between the upper wall 372 and the lower wall 374. A concavely curved side wall 378 extends generally orthogonally between the upper wall 372 and the lower wall 374. Bracing walls 380, 382 extend between the side walls 376, 378, defining a pair of lobes. Apertures 384 extend through the side walls 376, 378. Apertures 386 extend through the upper wall 372 and the lower wall 374.

The device 370 includes a solid region or layer 388 bonded to porous regions or layers 390, 392. The solid layer 388 may be formed of any of the biocompatible materials listed above for solid layer 46, including metals, polymers, ceramics, or composite materials. The solid layer 388 may be a uniform mass of material or may be a composite material having low or no porosity. Generally, the solid layer 388 may provide durability and/or strength to the device 370.

The porous layers 390, 392 may also be formed of biocompatible materials including metals, polymers, ceramics, or composite materials, including the specific materials listed above for porous layers 48, 50. The biocompatible materials of the porous layers are suspended in a porous architecture such that interconnected voids are interspersed between particles of the material. Generally, the porous layer may serve as a scaffold, permitting bone in-growth and fusion. The porous layers 390, 392 may be formed and bonded to the solid layer 388 as described above for layers 48, 50. In this embodiment, the solid layer 388 extends through and forms a portion of the bracing walls 380, 382 and the side walls 376, 378. The porous layers 390, 392 may form all or a portion of the upper and lower walls 372, 374, respectively.

The device 370 may be implanted using the methods described above for device 30. The apertures 384, 386 may be filled with filler material such as bone graft. The filler material together with the porous regions 390, 392 promote tissue in-growth. After the insertion of the device 370 between the vertebral bodies 12, 14 has been completed, the device 370 may promote the fusion or joining together of the vertebral bodies 12, 14.

In alternative embodiments, the upper and lower walls may be angled relative to one another to achieve a desired kyphosis, lordosis, or lateral wedge effect. In still other embodiments, a wall may extend obliquely from an adjacent wall rather than orthogonally. In still other alternative embodiments, the device may include geometry for mating with insertion or revision instruments.

Referring now to FIGS. 30 and 31, in another embodiment the intervertebral device 22 may be a device 400 substantially similar to the device 370, with the exceptions noted below. The device 400 includes an upper wall 402 and a lower wall 404, both of which have a generally crescent or boomerang shape. A convexly curved side wall 406 extends generally orthogonally between the upper wall 402 and the lower wall 404. A concavely curved side wall 408 extends generally orthogonally between the upper wall 372 and the lower wall 374. Both of the curved side walls 406, 408 may include indentions or protrusions. Bracing walls 410, 412 extend between the side walls 406, 408, defining a pair of lobes, 414, 416. Apertures 418 extend through the upper wall 402 and the lower wall 404.

The device 400 includes a solid region or layer 420 bonded to porous regions or layers 422, 424. The solid layer 420 may be formed of any of the biocompatible materials listed above for solid layer 46, including metals, polymers, ceramics, or composite materials. The solid layer 420 may be a uniform mass of material or may be a composite material having low or no porosity. Generally, the solid layer 420 may provide durability and/or strength to the device 400.

The porous layers 422, 424 may also be formed of biocompatible materials including metals, polymers, ceramics, or composite materials, including the specific materials listed above for porous layers 48, 50. The biocompatible materials of the porous layers are suspended in a porous architecture such that interconnected voids are interspersed between particles of the material. Generally, the porous layer may serve as a scaffold, permitting bone in-growth and fusion. The porous layers 422, 424 may be formed and bonded to the solid layer 420 as described above for layers 48, 50. In this embodiment, the solid layer 420 extends through and forms a portion of the bracing walls 410, 412 and the side walls 406, 408. The porous layers 422, 424 may form all or a portion of the upper and lower walls 402, 404, respectively. In an alternative embodiment, the walls 410, 412, 406, 408 defining the apertures 418 may be lined with a porous layer.

The device 400 may be implanted using the methods described above for device 30. The apertures 418 may be filled with filler material such as bone graft. The filler material together with the porous regions 422, 424 promote tissue in-growth. After the insertion of the device 400 between the vertebral bodies 12, 14 has been completed, the device 400 may promote the fusion or joining together of the vertebral bodies 12, 14.

Referring now to FIGS. 32 and 33, in another embodiment the intervertebral device 22 may be a device 430 substantially similar to the device 370, with the exceptions noted below. The device 430 includes an upper wall 432 and a lower wall 434, both of which have a generally crescent or boomerang shape. A convexly curved side wall 436 extends generally orthogonally between the upper wall 432 and the lower wall 434. A concavely curved side wall 438 extends generally orthogonally between the upper wall 432 and the lower wall 434. Bracing walls 440, 442 extend between the side walls 436, 438.

In this embodiment, a solid region 444 extends throughout and forms at least a portion of the walls, 432, 434, 436, 438, 440, 442. Recesses 446, 448 may be formed in the solid region 444 of the upper and lower walls 432, 434, respectively. The recesses 446, 448 may be filled with porous layers 450, 452. With the device 430 implanted and packed with filler material as described above, the porous regions 450, 452 may promote bone ingrowth to secure the device 430 within the disc space 20. In an alternative embodiment, the porous layers 450, 452 may extend outside of the recesses 446, 448 respectively and beyond the solid region 444 of the upper and lower walls.

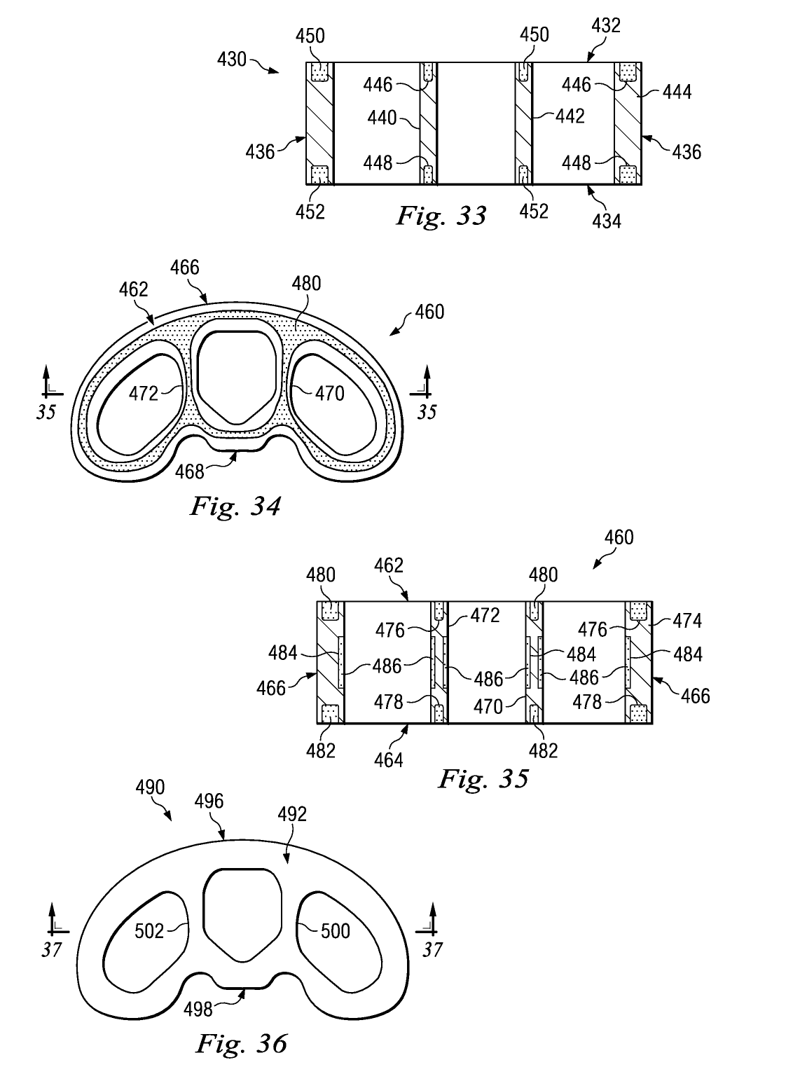

Referring now to FIGS. 34 and 35, in another embodiment the intervertebral device 22 may be a device 460 substantially similar to the device 430, with the exceptions noted below. The device 460 includes an upper wall 462 and a lower wall 464, both of which have a generally crescent or boomerang shape. A convexly curved side wall 466 extends generally orthogonally between the upper wall 462 and the lower wall 464. A concavely curved side wall 468 extends generally orthogonally between the upper wall 462 and the lower wall 464. Bracing walls 470, 472 extend between the side walls 466, 468.

In this embodiment, a solid region 474 extends throughout the walls, 462, 464, 466, 468, 470, 472. Recesses 476, 478 may be formed in the solid region 474 of the upper and lower walls 462, 464, respectively. The recesses 476, 478 may be filled with porous layers 480, 482. In this embodiment, additional recesses 484 may be formed in the solid region 474 of the bracing walls 470, 472 and the side walls 466, 468. The recesses 484 may be filled with a porous layer 486. With the device 460 implanted and packed with filler material as described above, the porous regions 480, 482, 486 may promote bone ingrowth to secure the device 460 within the disc space 20. In an alternative embodiment, the porous layers 480, 482 may extend outside of the recesses 476, 478 respectively and beyond the solid region 474 of the upper and lower walls.

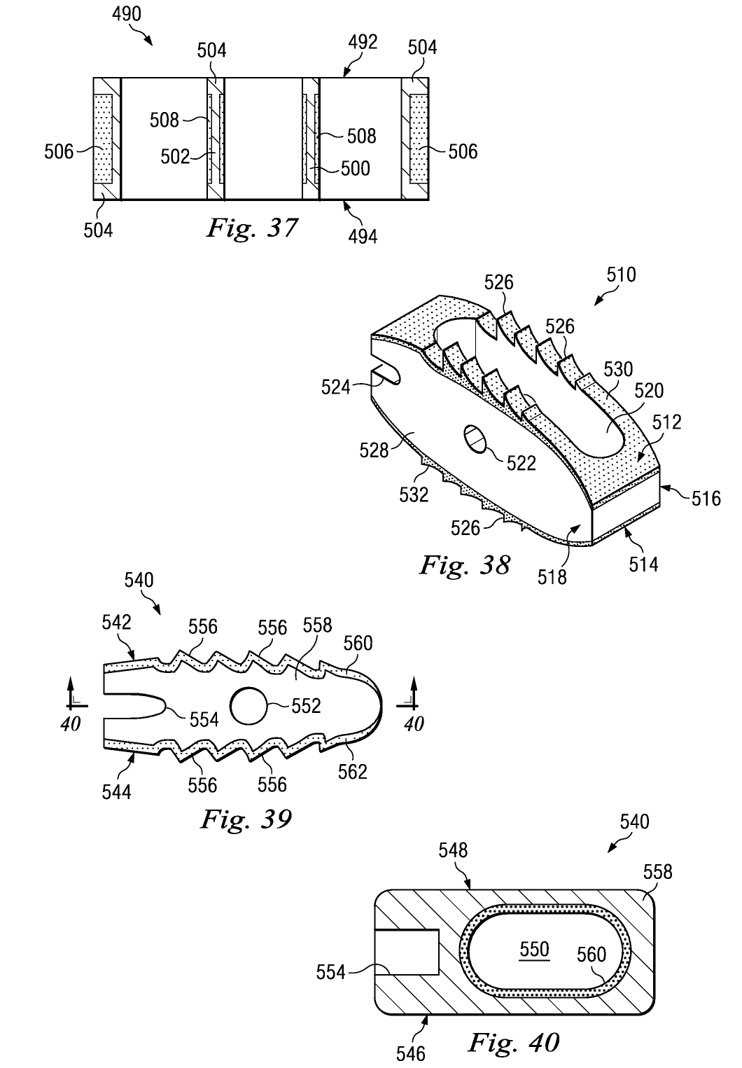

Referring now to FIGS. 36 and 37, in another embodiment the intervertebral device 22 may be a device 490 substantially similar to the device 430, with the exceptions noted below. The device 490 includes an upper wall 492 and a lower wall 494, both of which have a generally crescent or boomerang shape. A convexly curved side wall 496 extends generally orthogonally between the upper wall 492 and the lower wall 494. A concavely curved side wall 498 extends generally orthogonally between the upper wall 492 and the lower wall 494. Bracing walls 500, 502 extend between the side walls 496, 498.

In this embodiment, a solid region 504 extends throughout and forms at least a portion of the walls, 492, 494, 496, 498, 500, 502. A porous region 506 may extend through and form at least a portion of the side walls 496, 498, and a porous region 508 may extend through and form at least a portion of the bracing walls 500, 502. The porous regions 506, 508 may be connected at the juncture of the bracing walls and the side walls such that the porous regions 506, 508 are continuous. With the device 490 implanted and packed with filler material as described above, the porous regions 508, 506 may promote bone in-growth to secure the device 490 within the disc space 20.

Referring now to FIG. 38, in another embodiment the intervertebral device 22 may be an intervertebral fusion device referred to by the reference numeral 510. The device 510 may have a similar geometry to the CAPSTONE® vertebral spacer (distributed by or in development with Medtronic, Inc. of Minneapolis, Minn.) as described, at least in part, in U.S. Pat. Nos. 6,991,654 and 5,797,909 which are incorporated by reference herein.

The device 370 includes an arched upper wall 512 and an arched lower wall 514, forming a generally bullet shaped device that provides distraction for insertion into the disc space 20. Generally parallel side walls 516, 518 extend between the upper and lower walls 512, 514. The side walls 516, 518 and the upper and lower walls 512, 514 define a through aperture 520. Additional apertures 522 extend through the side walls 516, 518. Instrument attachment features 524 may be used for insertion, positioning, and/or revision of the device 510. The features 524 and the apertures 520, 522 may further serve to promote bone in-growth. The upper and lower walls 512, 514 may further include projections 526 to provide initial stability for the implanted device 510.

The device 510 includes a solid region or layer 528 bonded to porous regions or layers 530, 532. The solid layer 528 may be formed of any of the biocompatible materials listed above for solid layer 46, including metals, polymers, ceramics, or composite materials. The solid layer 528 may be a uniform mass of material or may be a composite material having low or no porosity. Generally, the solid layer 528 may provide durability and/or strength to the device 510.

The porous layers 530, 532 may also be formed of biocompatible materials including metals, polymers, ceramics, or composite materials, including the specific materials listed above for porous layers 48, 50. The biocompatible materials of the porous layers are suspended in a porous architecture such that interconnected voids are interspersed between particles of the material. Generally, the porous layer may serve as a scaffold, permitting bone in-growth and fusion. The porous layers 530, 532 may be formed and bonded to the solid layer 528 as described above for layers 48, 50. In this embodiment, the solid layer 528 extends through and forms a portion of the side walls 516, 518 and the upper and lower walls 512, 514. The porous layers 530, 532 may form a portion of the upper and lower walls 512, 514, respectively. In alternative embodiments, the porous layers may form all of the upper and lower walls.

The device 510 may be implanted using the methods described above for device 30. The apertures 520, 522 may be filled with filler material such as bone graft. The filler material together with the porous regions 530, 532 promote tissue growth into the device 510. After the insertion of the device 510 between the vertebral bodies 12, 14 has been completed, the device 510 may promote the fusion or joining together of the vertebral bodies 12, 14.

In alternative embodiments, the upper and lower walls may be angled relative to one another to achieve a desired kyphosis, lordosis, or lateral wedge effect. In still other embodiments, a wall may extend obliquely from an adjacent wall rather than orthogonally. In still other alternative embodiments, the device may include geometry for mating with insertion or revision instruments.

Referring now to FIGS. 39 and 40, in another embodiment the intervertebral device 22 may be a device 540 substantially similar to the device 510, with the exceptions noted below. The device 540 includes an arched upper wall 542 and an arched lower wall 544, forming a generally bullet shaped device that provides distraction for insertion into the disc space 20. Generally parallel side walls 546, 548 extend between the upper and lower walls 542, 544. The side walls 546, 548 and the upper and lower walls 542, 544 define a through aperture 550. Additional apertures 552 extend through the side walls 546, 548. Instrument attachment features 554 may be used for insertion, positioning, and/or revision of the device 540. The features 554 and the apertures 550, 552 may further serve to promote bone in-growth. The upper and lower walls 542, 544 may further include projections 556 to provide initial stability for the implanted device 540.

The device 540 includes a solid region or layer 558 bonded to porous regions or layers 560, 562. The porous regions 560, 562 comprise all or a portion of the upper and lower walls 542, 544 and extend to layer the surfaces defining the aperture 550. With porous layers thusly configured, bone growth into the device 540 and through the aperture 550 is promoted, thereby promoting fusion of the joint 10.

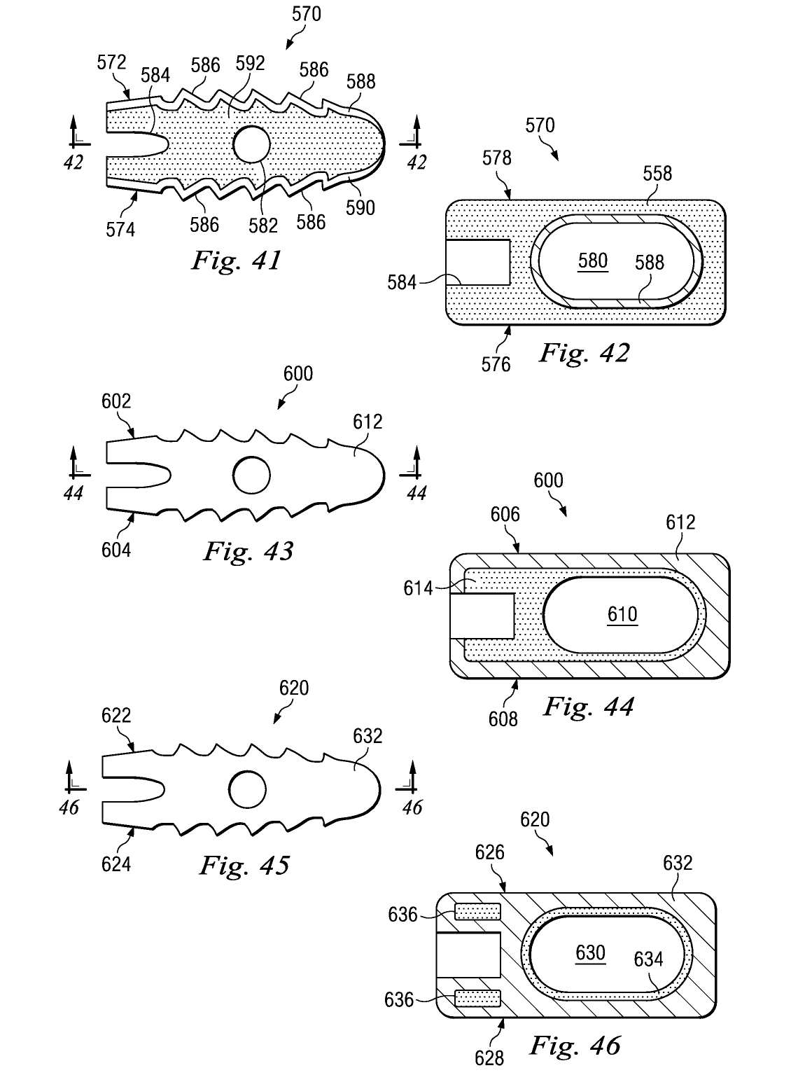

Referring now to FIGS. 41 and 42, in another embodiment the intervertebral device 22 may be a device 570 substantially similar to the device 540, with the exceptions noted below. The device 570 includes an arched upper wall 572 and an arched lower wall 574, forming a generally bullet shaped device that provides distraction for insertion into the disc space 20. Generally parallel side walls 576, 578 extend between the upper and lower walls 572, 574. The side walls 576, 578 and the upper and lower walls 572, 574 define a through aperture 580. Additional apertures 582 extend through the side walls 576, 578. Instrument attachment features 584 may be used for insertion, positioning, and/or revision of the device 570. The features 584 and the apertures 580, 582 may further serve to promote bone in-growth. The upper and lower walls 572, 574 may further include projections 586 to provide initial stability for the implanted device 570.

The device 570 includes solid regions or layers 558, 590 bonded to a porous region 592. The solid regions 588, 590 comprise all or a portion of the upper and lower walls 572, 574. The porous region 592 extends throughout and forms at least a portion of the side walls 576, 578 and defines at least a portion of the aperture 580. With the porous layer thusly configured, bone growth into the device 570 and through the aperture 580 is promoted, thereby promoting fusion of the joint 10.

Referring now to FIGS. 43 and 44, in another embodiment the intervertebral device 22 may be a device 600 substantially similar to the device 540, with the exceptions noted below. The device 600 includes an arched upper wall 602 and an arched lower wall 604, forming a generally bullet shaped device that provides distraction for insertion into the disc space 20. Generally parallel side walls 606, 608 extend between the upper and lower walls 602, 604. The side walls 606, 608 and the upper and lower walls 602, 604 define a through aperture 610.

The device 600 includes solid regions 612 bonded to porous regions 614. The solid regions 612 form a portion of the upper and lower walls 602, 604. The porous region 614 extends throughout at least a portion of the side walls 606, 608 and defines at least a portion of the aperture 610. The porous region 614 may extend along the aperture 610 and form the openings to the aperture 610 in the upper and lower walls 602, 604. The porous region 614 may further extend to form instrument attachment features. With the porous layer configured as described, bone growth into the device 600 and through the aperture 610 is promoted, thereby promoting fusion of the joint 10.

Referring now to FIGS. 45 and 46, in another embodiment the intervertebral device 22 may be a device 620 substantially similar to the device 600, with the exceptions noted below. The device 620 includes an arched upper wall 622 and an arched lower wall 624, forming a generally bullet shaped device that provides distraction for insertion into the disc space 20. Generally parallel side walls 626, 628 extend between the upper and lower walls 622, 624. The side walls 626, 628 and the upper and lower walls 622, 624 define a through aperture 630.

The device 620 includes solid regions 632 bonded to porous regions 634, 636. The solid regions 632 form a portion of the upper and lower walls 622, 624. The porous region 634 extends throughout at least a portion of the side walls 606, 608 and defines at least a portion of the aperture 630. The porous region 634 may extend along the aperture 630 and form the openings to the aperture 630 in the upper and lower walls 62, 624. The additional porous regions 636 may extend through the solid region 632 and form portions of the upper and lower walls 622, 624. The porous regions 636 may be discontinuous with the porous region 634. With the porous regions configured as described, bone growth into the device 620 and through the aperture 630 is promoted, thereby promoting fusion of the joint 10.

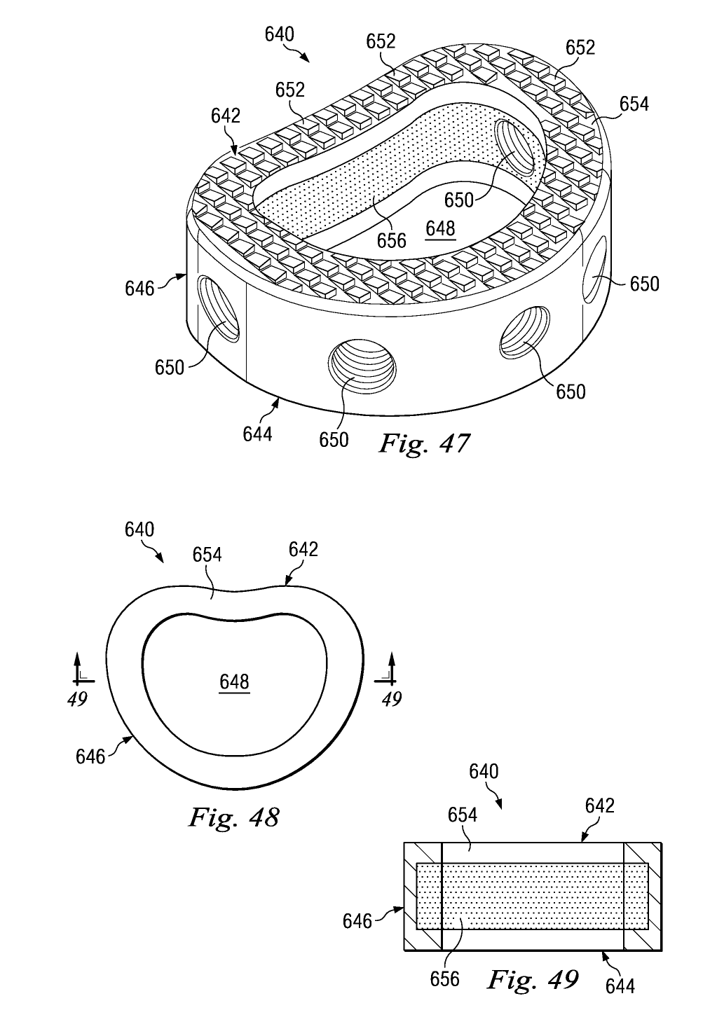

Referring now to FIGS. 47, 48, and 49, in another embodiment the intervertebral device 22 may be an intervertebral fusion device referred to by the reference numeral 640. The device 640 may have a similar geometry to the PERIMETER® vertebral spacers (distributed by or in development with Medtronic, Inc. of Minneapolis, Minn.). The device 640 is a generally D-shaped ring with a shape that tends to match the natural geometry of an intervertebral annulus and/or the footprint of a vertebral body. The device 640 includes an upper wall 642 and a lower wall 644. A side wall 646 extends between the upper wall and the lower walls 642, 644. In this embodiment, the upper and lower walls 642, 644 are generally parallel, but in alternative embodiments, the upper and lower walls may be angled to create a desired kyphotic or lordotic angle in the intervertebral device.

The upper, lower, and side walls define an aperture 648. Additional apertures 650 extend through the side walls 646. The apertures 650 may serve as instrument attachment features for insertion, positioning, and/or revision of the device 650 and/or may serve as bone in-growth portals. The device 640 may further include projections 652 extending from the upper and lower walls 642, 644.

The device 640 includes a solid region or layer 654 bonded to a porous region or layer 656. The solid layer 654 may be formed of any of the biocompatible materials listed above for solid layer 46, including metals, polymers, ceramics, or composite materials. The solid layer 654 may be a uniform mass of material or may be a composite material having low or no porosity. Generally, the solid layer 654 may provide durability and/or strength to the device 640.

The porous region 656 may also be formed of biocompatible materials including metals, polymers, ceramics, or composite materials, including the specific materials listed above for porous layers 48, 50. The biocompatible materials of the porous layers are suspended in a porous architecture such that interconnected voids are interspersed between particles of the material. Generally, the porous layer may serve as a scaffold, permitting bone in-growth and fusion. The porous region 656 may be formed and bonded to the solid layer 654 as described above for layers 48, 50. In this embodiment, the porous region 656 defines a portion of the aperture 648.

The device 640 may be implanted using the methods described above for device 30. The apertures 648, 650 may be filled with filler material such as bone graft or any of the filler materials described above. The filler material together with the porous regions 656 promote tissue growth into the device 640. After the insertion of the device 640 between the vertebral bodies 12, 14 has been completed, the device 640 may promote the fusion or joining together of the vertebral bodies 12, 14.

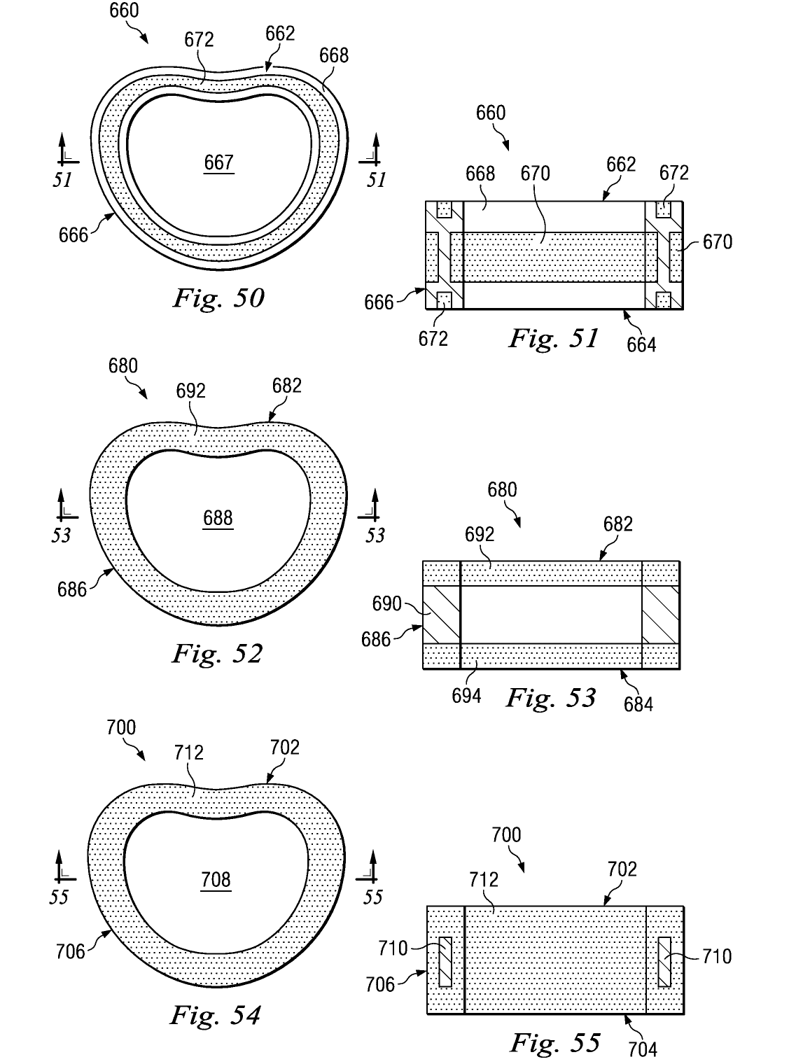

Referring now to FIGS. 50 and 51, in another ring shaped embodiment, an intervertebral device 660 may include an upper wall 662 and a lower wall 664. A side wall 666 extends between the upper wall and the lower walls 662, 664. In this embodiment, the upper and lower walls 662, 664 are generally parallel, but in alternative embodiments, the upper and lower walls may be angled to create a desired kyphotic or lordotic angle in the intervertebral device. The upper, lower, and side walls define an aperture 667.

The device 660 includes a solid region or layer 668 bonded to porous regions or layers 670, 672. In this embodiment, the porous regions 672 extend into the upper and lower walls 662, 664. The porous regions 670 extend into the side wall 666. The porous regions 670, 672 may form at least four discrete regions within the device 660 or the regions may be connected at various locations to form a continuous porous region. The device 660 maybe packed with filler material and implanted as described above. The filler material together with the porous regions 670, 672 promote tissue growth into the device 660. After the insertion of the device 660 between the vertebral bodies 12, 14 has been completed, the device 660 may promote the fusion or joining together of the vertebral bodies 12, 14.

Referring now to FIGS. 52 and 53, in another ring shaped embodiment, an intervertebral device 680 may include an upper wall 682 and a lower wall 684. A side wall 686 extends generally orthogonally between the upper wall and the lower walls 682, 684. In this embodiment, the upper and lower walls 682, 684 are generally parallel, but in alternative embodiments, the upper and lower walls may be angled to create a desired kyphotic or lordotic angle in the intervertebral device. The upper, lower, and side walls define an aperture 688.

The device 680 includes a solid region or layer 690 bonded to porous regions or layers 692, 694. In this layered embodiment, the solid region 690 extends between the porous layers 692, 694. The device 680 may be packed with filler material and implanted as described above. The filler material together with the porous regions 682, 684 promotes tissue growth into the device 680. After the insertion of the device 680 between the vertebral bodies 12, 14 has been completed, the device 680 may promote the fusion or joining together of the vertebral bodies 12, 14.

Referring now to FIGS. 54 and 55, in another ring shaped embodiment, an intervertebral device 700 may include an upper wall 702 and a lower wall 704. A side wall 706 extends between the upper and lower walls 702, 704. In this embodiment, the upper and lower walls 702, 704 are generally parallel, but in alternative embodiments, the upper and lower walls may be angled to create a desired kyphotic or lordotic angle in the intervertebral device. The upper, lower, and side walls define an aperture 708.

The device 700 includes a solid region or layer 710 bonded to porous regions or layers 712. In this embodiment, the solid region 710 is encapsulated by the porous region 712. The device 700 maybe packed with filler material and implanted as described above. The filler material together with the porous regions 712 promotes tissue growth into the device 700. After the insertion of the device 700 between the vertebral bodies 12, 14 has been completed, the device 700 may promote the fusion or joining together of the vertebral bodies 12, 14.

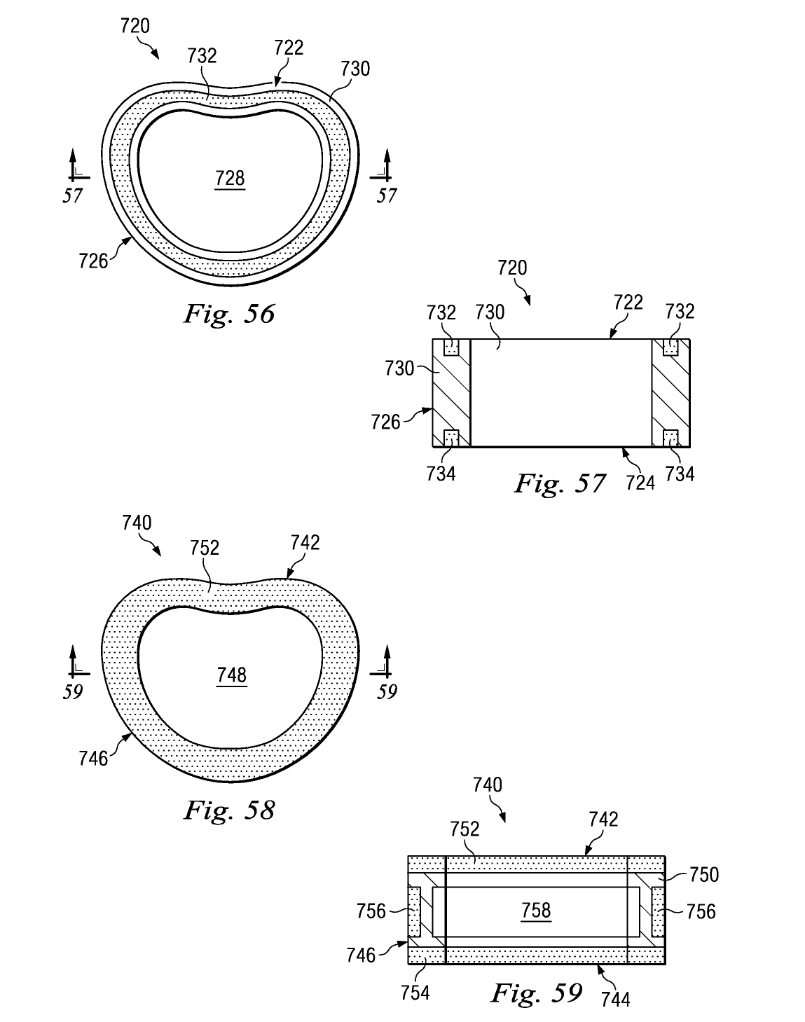

Referring now to FIGS. 56 and 57, in another ring shaped embodiment, an intervertebral device 720 may include an upper wall 722 and a lower wall 724. A side wall 726 extends between the upper and lower walls 722, 724. In this embodiment, the upper and lower walls 722, 724 are generally parallel, but in alternative embodiments, the upper and lower walls may be angled to create a desired kyphotic or lordotic angle in the intervertebral device. The upper, lower, and side walls define an aperture 728.

The device 720 includes a solid region or layer 730 bonded to porous regions 732, 734. In this embodiment, the porous region 732, 734 extend into the upper and lower walls 662, 664, respectively. The porous regions 732, 734 fill recesses in the solid region 730. The device 720 may be packed with filler material and implanted as described above. The filler material together with the porous regions 732, 734 promotes tissue growth into the device 720. After the insertion of the device 720 between the vertebral bodies 12, 14 has been completed, the device 720 may promote the fusion or joining together of the vertebral bodies 12, 14.

Referring now to FIGS. 58 and 59, in another ring shaped embodiment, an intervertebral device 740 may include an upper wall 742 and a lower wall 744. A side wall 746 extends between the upper and lower walls 742, 744. In this embodiment, the upper and lower walls 742, 744 are generally parallel, but in alternative embodiments, the upper and lower walls may be angled to create a desired kyphotic or lordotic angle in the intervertebral device. The upper, lower, and side walls define an aperture 748.

The device 740 includes a solid region or layer 750 bonded to porous regions or layers 752, 754, 756, 758. In this embodiment, the porous regions 752, 754 extend across the solid regions 750, forming all or a portion of the upper and lower walls 742, 744, respectively. The porous regions 756, 758 extend into the side wall 746. The porous regions 752, 754, 756, 758 may form at least four discrete regions within the device 740 or the regions may be connected at various locations to form a continuous porous region. The device 740 maybe packed with filler material and implanted as described above. The filler material together with the porous regions 752, 754, 756, 758 promote tissue growth into the device 740. After the insertion of the device 740 between the vertebral bodies 12, 14 has been completed, the device 740 may promote the fusion or joining together of the vertebral bodies 12, 14.

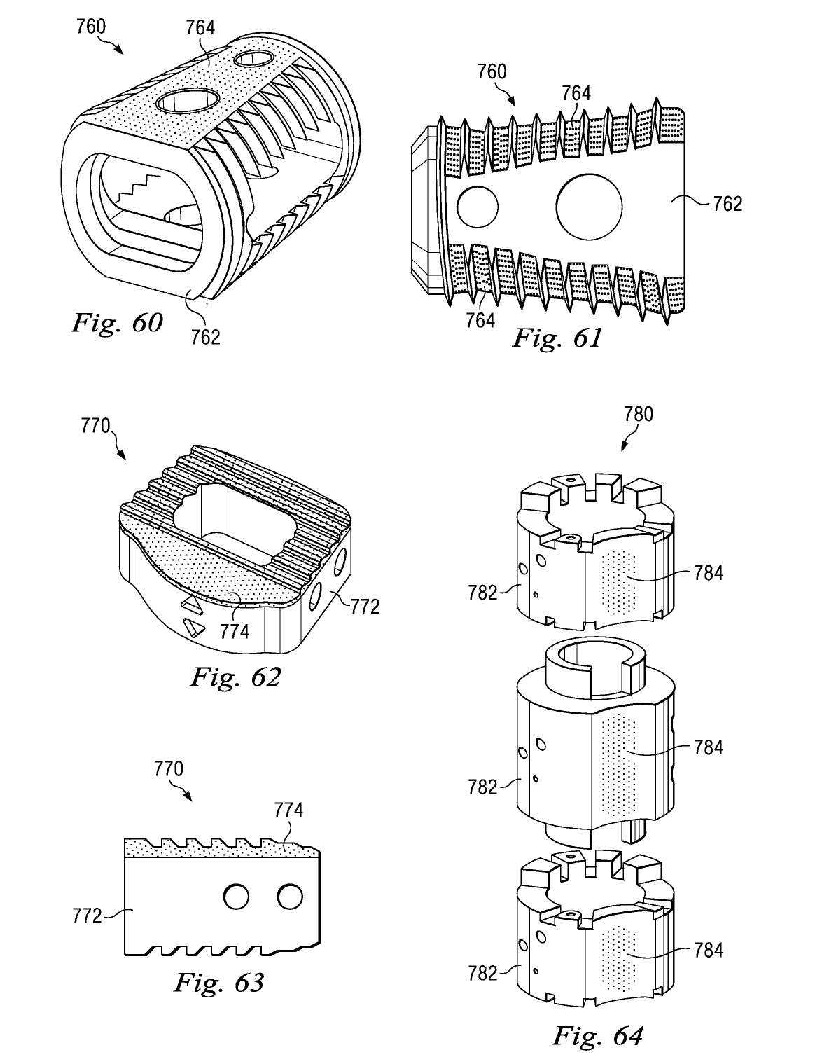

The use of porous layers or regions bonded on non-porous layers or regions may be applied to any of a number of fusion and motion preserving intervertebral devices, including those distributed by or in development with Medtronic, Inc. of Minneapolis, Minn. Referring now to FIGS. 60 and 61, a device 760 may be the same or substantially similar to a Medtronic LT-Cage® device which may be covered at least in part by the following U.S. Patents which are incorporated herein by reference: U.S. Pat. Nos. 5,645,549; 5,669,909; 5,782,919; 5,785,707; 5,984,967; 6,206,922; 6,245,072; 6,375,655; 6,471,724; 6,595,995; 6,613,091; 6,645,206; and 6,695,851. The device 760 may include solid regions 762 fused to porous regions 764. The solid and porous regions 762, 764 may be formed, fused, and utilized as described above for the solid 46 and porous 48, 50 regions described above.

Referring now to FIGS. 62 and 63, a device 770 may be the same or substantially similar to a Medtronic Cornerstone® device which may be covered at least in part by the following U.S. Patents which are incorporated herein by reference: U.S. Pat. Nos. 6,758,862 and 6,991,653. The device 770 may include solid regions 772 fused to porous regions 774. The solid and porous regions 772, 774 may be formed, fused, and utilized as described above for the solid 46 and porous 48, 50 regions described above.

Referring now to FIG. 64, a device 780 may be the same or substantially similar to a modular Medtronic Verte-Stack® device which may be covered at least in part by the following U.S. Patents which are incorporated herein by reference: U.S. Pat. No. 6,758,862 and 6,991,653. The device 780 may include solid regions 782 fused to porous regions 784. The solid and porous regions 782, 784 may be formed, fused, and utilized as described above for the solid 46 and porous 48, 50 regions described above.

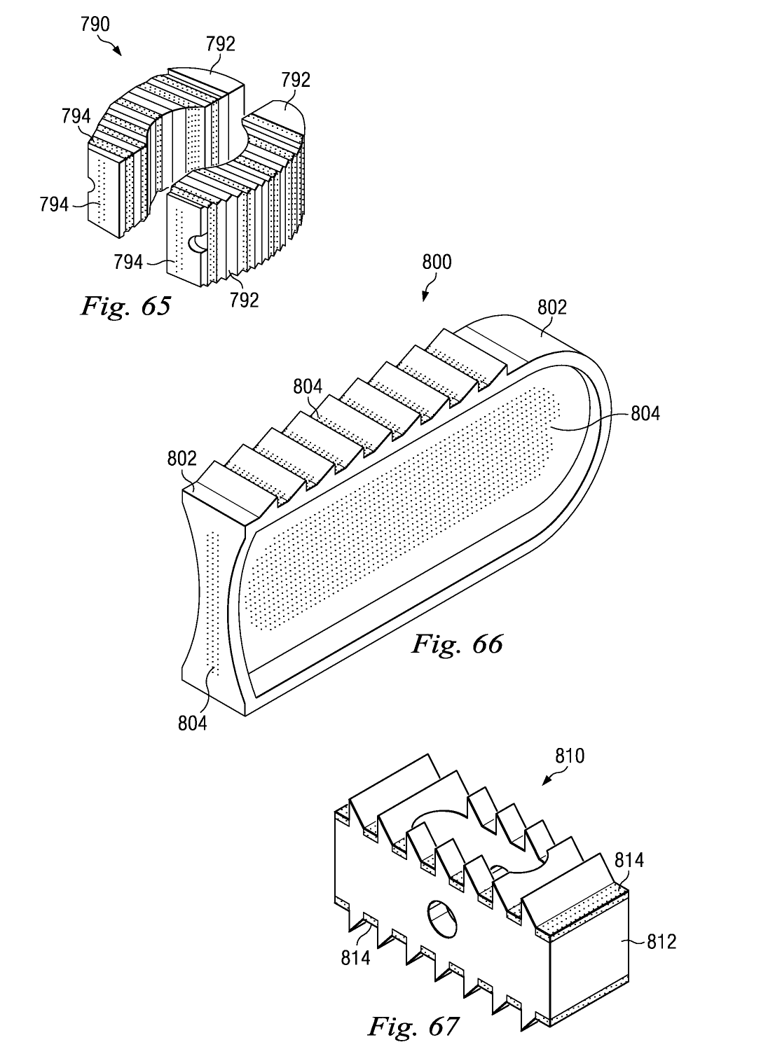

Referring now to FIG. 65, a device 790 may be the same or substantially similar to a Medtronic Tangent® device which may be covered at least in part by the following U.S. Patents which are incorporated herein by reference: U.S. Pat. Nos. 6,174,311; 6,610,065; 6,610,089; 6,096,038; and 5,797,909. The device 790 may include solid regions 792 fused to porous regions 794. The solid and porous regions may be formed, fused, and utilized as described above for the solid 46 and porous 48, 50 regions described above.

Referring now to FIG. 66, a device 800 may be the same or substantially similar to a Medtronic Hourglass® Vertebral Body Spacer which may be covered at least in part by U.S. patent application Ser. No. 10/404,262 which is incorporated by reference herein. The device 800 may include solid regions 802 fused to porous regions 804. The solid and porous regions 802, 804 may be formed, fused, and utilized as described above for the solid 46 and porous 48, 50 regions described above.

Referring now to FIG. 67, a device 810 may be the same or substantially similar to a Medtronic Telamon® device which may be covered at least in part by the following U.S. Patent which is incorporated herein by reference, U.S. Pat. No. 6,746,484. The device 810 may include solid regions 812 fused to porous regions 814. The solid and porous regions 812, 814 may be formed, fused, and utilized as described above for the solid 46 and porous 48, 50 regions described above.

Any or all of the porous regions or layers described above may incorporate a biologic material to encourage bone growth into or onto the device. Suitable biologic materials may include osteoconductive material such as hydroxyapatite (HA), tricalcium phosphate (TCP), amorphous calcium phosphate (ACP) and/or calcium carbonate. Alternatively, osteoinductive coatings, such as proteins from transforming growth factor (TGF) beta superfamily, or bone-morphogenic proteins, such as BMP2 or BMP7, may be used.

It is understood that the use of porous layers bonded to non porous layers may be used for intervertebral devices other than fusion devices. For example, a motion preservation implant such as those described in U.S. Pat. Nos. 6,740,118 or 6,156,067, which are incorporated by reference herein, may be configured such that porous layers are bonded to the rigid endplates or shells disclosed. In this embodiment, the porous layers will serve to promote tissue growth into the endplates or shells to secure the motion preserving implant within the disc space.

Although only a few exemplary embodiments have been described in detail above, those skilled in the art will readily appreciate that many modifications are possible in the exemplary embodiments without materially departing from the novel teachings and advantages of this disclosure. Accordingly, all such modifications and alternative are intended to be included within the scope of the invention as defined in the following claims. Those skilled in the art should also realize that such modifications and equivalent constructions or methods do not depart from the spirit and scope of the present disclosure, and that they may make various changes, substitutions, and alterations herein without departing from the spirit and scope of the present disclosure. It is understood that all spatial references, such as “horizontal,” “vertical,” “top”, “upper,” “lower,” “bottom,” “left,” “right,” “anterior,”“posterior”, “superior,” “inferior,” “upper,” and “lower” are for illustrative purposes only and can be varied within the scope of the disclosure. In the claims, means-plus-function clauses are intended to cover the elements described herein as performing the recited function and not only structural equivalents, but also equivalent elements.

1. A device for promoting fusion of first and second vertebrae, the device comprising;
a first solid region formed of non-porous polyetheretherketone (PEEK) and
a first porous region including a porous PEEK architecture,
wherein the first porous region is bonded to the first solid region.

2. The device of claim 1 wherein the porous PEEK architecture comprises a plurality of sintered PEEK particles.

3. The device of claim 1 wherein the first porous region is heated to bond to the first solid region.

4. The device of claim 1 wherein the first porous region covers all surfaces of the first solid region.

5. The device of claim 1 wherein the porous PEEK architecture comprises a plurality of machined voids.

6. The device of claim 1 wherein the porous PEEK architecture comprises a plurality of chemically etched voids.

7. The device of claim 1 wherein the first porous region covers a first surface of the first solid region and a second porous region, discontinuous from the first porous region, covers a second surface of the first solid region.

8. The device of claim 1 further comprising a plurality of projections integrally formed with and extending from the first solid region wherein the plurality of projections protrude through the first porous region.

9. The device of claim 1 wherein the first porous region includes a plurality of discrete regions of sintered PEEK particles and further wherein the discrete regions are separated by non-porous regions of PEEK.

10. The device of claim 1 wherein the first solid region includes a recessed surface and the first porous region covers the recessed surface.

11. The device of claim 10 wherein the first solid region includes an outer surface in a generally parallel but separate plane than the recessed surface and wherein the first porous region continuously covers the outer surface and the recessed surface.

12. The device of claim 1 further including first and second walls adapted to extend between the first and second vertebrae and third and fourth walls connected to and extending generally orthogonally from the first and second walls, wherein the first wall includes the first solid region formed of non-porous PEEK.

13. The device of claim 12 wherein the second, third and fourth walls also include second, third, and fourth solid regions, respectively, formed of non-porous PEEK.

14. The device of claim 13 wherein at least two of the first, second, third, and fourth solid regions are directly connected.

15. The device of claim 12 wherein the third wall includes the first porous region and the fourth wall includes a second porous region and further wherein the third wall is adapted to interface with the first vertebra and the fourth wall is adapted to interface with the second vertebra.

16. The device of claim 1 further comprising upper and lower generally crescent shaped walls interconnected at least in part by a convexly curved outer wall and a concavely curved inner wall, wherein the upper wall includes the first porous region.

17. The device of claim 16 wherein at least one aperture extends through the inner and outer walls.

18. The device of claim 16 wherein the upper wall includes a recessed channel and the first porous region extends within and along the recessed channel.

19. The device of claim 16 wherein the outer wall includes a recessed channel and the first porous layer extend within the recessed channel

20. The device of claim 16 wherein a bracing wall extends between the outer and inner walls.

21. The device of claim 20 wherein the first solid region forms at least a portion of the outer, inner, and bracing walls.

22. The device of claim 20 wherein the first solid region forms at least a portion of the outer wall and a second solid PEEK region forms at least a portion of the bracing wall, wherein the first and second solid regions are connected by the first porous region.

23. The device of claim 20 wherein the bracing wall includes at least a portion of the first porous region.

24. The device of claim 20 wherein the bracing wall includes a recessed channel and the first porous region extends within and along the recessed channel.

25. The device of claim 1 further comprising a through passage in the first solid region.

26. The device of claim 25 wherein the passage defines an inner surface of the first solid region and further wherein the first porous region covers at least a portion of the inner surface.

27. The device of claim 25 wherein the first porous region fills the passage through the first solid region.

28. The device of claim 1 wherein the first solid region forms an annulus shaped ring having a perimeter surface, an inner surface, and an upper surface opposite a lower surface.

29. The device of claim 28 wherein the first porous region is deposited in a channel in the first solid region, the channel extending into the inner surface of the ring.

30. The device of claim 28 wherein the first porous region is deposited in a channel in the first solid region, the channel extending into the perimeter surface of the ring.

31. The device of claim 28 wherein the first porous region is deposited in a channel in the first solid region, the channel extending into the upper surface of the ring.

32. The device of claim 28 wherein the first porous region is deposited in a channel in the first solid region, the channel extending into the lower surface of the ring.

33. The device of claim 28 wherein the first porous region covers the upper surface.

34. The device of claim 33 wherein the first porous region covers the lower surface.

35. The device of claim 33 wherein the first porous region covers the lower, inner and perimeter surfaces, encapsulating the first solid region.

36. The device of claim 1 wherein the first solid region includes a pair of opposite curved surfaces interconnected by a pair of opposite generally flat and wedge shaped surfaces.

37. The device of claim 1 wherein the first porous region has a thickness between approximately 0.5 and 3.0 mm.

38. The device of claim 1 wherein the first porous region has a tapered thickness.

39. The device of claim 1 wherein the first porous region includes at least one ridge.

40. The device of claim 39 wherein the at least one ridge is formed by machining the first porous region.

41. The device of claim 39 wherein the at least one ridge is formed by molding the first porous layer.

42. The device of claim 1 further comprising
a second solid region formed of non-porous PEEK and
a second porous region including a porous PEEK architecture,
wherein the second porous layer is bonded to the second solid region such that the second porous region covers at least a portion of one surface of the second solid region and further wherein the second solid region is adapted to mechanically connect to the first solid region.

43. The device of claim 42 wherein the first solid region comprises an extension and the second solid region comprises an opening and wherein the extension is adapted for insertion into the opening.

44. The device of claim 1 wherein the first solid region includes a pair of opposite concave surfaces.

45. The device of claim 1 further including a bone growth promoting additive applied to the first porous region.

46. The device of claim 45 wherein the bone growth promoting additive is hydroxyapatite.

47. The device of claim 45 wherein the bone growth promoting additive includes a bone morphogenic protein.

48. The device of claim 45 wherein the bone growth promoting additive includes amorphous calcium phosphate.

49. The device of claim 1 wherein the first porous region is bonded to the first solid region with an adhesive.

50. A surgical method comprising:
removing at least a portion of an intervertebral disc from between a pair of vertebral bodies to create an intervertebral space;
selecting an a device comprising a solid region including a non-porous polymer and a porous region including a porous polymer architecture, wherein the porous region is bonded to the solid region;
inserting the device into the intervertebral space to promote bone growth between the pair of vertebral bodies.

51. The surgical method of claim 50 wherein the non-porous polymer includes PEEK and the porous polymer architecture includes PEEK.

52. The surgical method of claim 50 wherein the step of inserting the device includes positioning the device such that the first porous region abuts at least one of the pair of vertebral bodies.

53. The surgical method of claim 50 further comprising: resecting at least a portion of an endplate of one of the pair of vertebral bodies to create a resected area.

54. The surgical method of claim 53 wherein the step of inserting further includes positioning at least a portion of the device into the resected area.

55. The surgical method of claim 50 wherein the step of inserting the device includes inserting the device through a generally anterior approach.

56. The surgical method of claim 50 wherein the step of inserting the device includes inserting the device through a generally posterior approach.

57. An apparatus comprising:
a first wall adapted to engage a first vertebral endplate;
a second wall adapted to engage a second vertebral endplate;
a third wall extending generally orthogonally between the first and second walls;
a fourth wall extending generally orthogonally between the first and second walls and spaced apart from the third wall; and
a cavity defined at least in part by the first, second, third, and fourth walls and adapted to receive a bone growth promoting material,
wherein at least one of the walls includes a solid region formed of non-porous first material and a scaffold region formed of a porous second material.

58. The apparatus of claim 57 wherein the first material is a metal and the second material is a polymer.

59. The apparatus of claim 57 wherein the first material is a polymer and the second material is a metal.

60. The apparatus of claim 57 wherein the first and second materials are both polymers.

61. The apparatus of claim 57 wherein at least one of the first and second materials is a ceramic.

62. The apparatus of claim 57 wherein the first wall is crescent shaped.

63. The apparatus of claim 57 wherein the first wall is ring shaped.

64. The apparatus of claim 57 wherein the third wall includes the solid region bonded to the scaffold region.

65. An apparatus for promoting bone growth between first and second bone portions, the device comprising:
a first solid layer formed of non-porous PEEK and
a first porous layer formed of PEEK particles,
wherein the PEEK particles are sintered together to form the first porous layer and further wherein the PEEK particles are fused to the non-porous PEEK of the first solid layer such that the first porous layer covers at least a portion of the first solid layer.

66. The apparatus of claim 65 wherein the first porous layer is fused to the first solid layer with a chemical adhesive.

67. The apparatus of claim 65 wherein the first porous layer is fused to the first solid layer by heating at least one of the layers.

68. The apparatus of claim 65 further comprising a second porous layer formed of PEEK particles, wherein the second porous layer is fused to the first solid layer.

69. The apparatus of claim 65 further comprising a second porous layer formed of PEEK particles, wherein the second porous layer is fused to the first porous layer, wherein the first porous layer has a first porosity and the second porous layer has a second porosity, and wherein the first and second porosities are different.
